(19) United States
(12) Patent Application Publication
(10) Pub. No.: US 2014/0234406 A1
(43) Pub. Date: Aug. 21, 2014

(54) NOVEL NANOPARTICULATE SUSPENSION FOR INCORPORATING FAT-SOLUBLE VITAMINS IN THE TREATMENT OF HAIR THINNING and HAIR LOSS

(71) Applicants: Stuart Lawrence Cantor, Mount Airy, MD (US); David Feder, Buffalo Grove, IL (US)

(72) Inventors: Stuart Lawrence Cantor, Mount Airy, MD (US); David Feder, Buffalo Grove, IL (US)

(73) Assignee: HairRx, Buffalo Grove, IL (US)

(21) Appl. No.: 13/769,326

(22) Filed: Feb. 17, 2013

(51) Int. Cl. A61K 33/00; A61K 31/375; A61K 9/48; A61K 31/07; A61K 45/06; A61K 31/593; A61K 31/355

(52) U.S. Cl. CPC A61K 33/00; A61K 31/593; A61K 31/375; A61K 31/355; A61K 31/07; A61K 45/06; A61K 9/48
USPC 424/451; 424/724

(57) ABSTRACT

The present invention provides novel compositions comprising a nutritional supplement containing vitamin D-3 (cholecalciferol) and other supporting nutrients (i.e., fat-soluble vitamins A and E) to be administered orally via capsules to human subjects for the prophylaxis and mitigation of hair thinning and hair loss in both men and women. Colloidal silicon dioxide functions to form a nanoparticulate suspension and acts as a viscosifier to thicken the oil(s) used in the capsule filling operation. The colloidal silica contains silicon which acts as a nutrient to help strengthen existing hair, and also functions as a microencapsulation agent, which can incorporate vitamins A and E into its porous structure by sonication.

References Cited [Referenced By]
U.S. Patent Documents

| | | |
|---|---|---|
| 4,749,684 | June 1988 | Silvestrini |
| 5,486,509 | January 1996 | Jimenez et al. |
| 5,744,128 | April 1998 | Holick |
| 5,958,384 | September 1999 | Holick |
| 6,149,933 | November 2000 | Nelson |
| 20070231377 | October 2007 | Abou-Nemeh |
| 7,452,527 | November 2008 | Murad |
| 20090041856 | February 2009 | Murad |
| 20100172993 | July 2010 | Singh et al. |
| 20110236493 | September 2011 | Canham et al. |
| 20110287061 | November 2011 | Beggan |
| 8,318,140 | November 2012 | Murad |
| 20120142598 | June 2012 | Zeev |

### FIELD OF THE INVENTION

The present invention provides formulations and methods useful as prophylaxis and mitigation of hair loss in either men or women. The formulations comprise certain concentrations of vitamin D3 and other associated important and supportive nutrients responsible for maintaining the health and integrity of the hair follicles and associated glands.

The other nutrients consist of two other fat-soluble vitamins, vitamin A and vitamin E. Both are microencapsulated into the pores of the silica nanoparticles. The silica has several roles in this formulation, it acts as a microencapsulating agent to entrap the fat-soluble vitamins, it acts as an oil viscosifier and an anti-settling agent, and it also serves as a nutrient to nourish the hair follicles.

The formulation can contain either fat-soluble nutrients or a combination of water-soluble and fat-soluble nutrients. If a combination product is produced, an emulsifier or surfactant would be added to ensure that the mixture is homogeneous.

### BACKGROUND OF THE INVENTION

U.S. Pat. No. 4,749,684 teaches a method of promoting linear hair growth that involves giving patients not less than 100 mg/kg, or 8-16 grams daily, of gelatin for at least three months. That patent discloses that lower dosages of gelatin are not sufficient to produce an effect on hair growth. Moreover, the gelatin, when administered as a capsule dosage form, was found to be irritating to the subjects, and was therefore provided in the form of yogurt.

U.S. Pat. No. 5,486,509 dated Jan. 23, 1996 taught methods of preventing and treating chemotherapy-induced alopecia by the prior administration of a growth factor, such as EGF, and vitamin D3 to Sprague-Dawley rats. The rats received the dosages either intraperitoneally, subcutaneously, or topically for a time period of between 4-6 days. Jimenez et al., found that a growth factor, such as EGF, and Vitamin D3 appear to render the hair follicle resistant to the toxic effect of chemotherapeutic agents thus preventing hair loss in rats.

U.S. Pat. Nos. 5,744,128 dated Apr. 28, 1998 and 5,958,384 dated Sep. 28, 1999, teach the use of emu oil in one embodiment for stimulating skin and hair growth. Holick also studied this preparation in a second embodiment comprising emu oil, or a biologically active fraction thereof, and one or more active vitamin D compounds in a preferred concentration level of 10 μg/g composition. However, the emu oil is derived from a bird indigenous only to Australia and is a compound that is not available in large supply. Furthermore, commercial emu oil products are not standardized, and vary widely in their potency. Three adolescent mice were administered the emu oil preparation, however, this sample size is too small to draw inferences regarding the beneficial effects of emu oil. Moreover, some can exhibit localized skin irritation as well as allergic reactions from the topical administration of said creams, pastes, and ointment preparations.

U.S. Pat. App. No. 20120142598 teaches edible compositions for preventing hair loss containing gelatin as the active agent and comprising between 5% to about 50% w/w of the formulation. However, the author discloses the fact that there is little hard scientific evidence to support the statements made for gelatin products. The author states further that the body already manufacturers abundant amounts of the amino acids that gelatin provides and that there is no solid experimental evidence to prove whether extra supplies of gelatin will help build healthy hair and nails.

U.S. Pat. App. No. 20090041856, and U.S. Pat. Nos. 7,452,527 and 8,318,140 by Murad teach the use of a combination of ingredients for the treatment of hair loss and to promote hair growth. These ingredients include an antioxidant in the form of vitamin C or a salt or ester of vitamin C, a transition metal consisting of zinc, manganese, copper, or mixtures thereof, vitamin B, vitamin E, one amino acid selected from the group consisting of proline, lysine, cysteine, and methionine, and a sugar compound that is converted to a glycosaminoglycan in the patient. In one embodiment, the sugar compound is N-acetyl glucosamine, D-glucosamine sulfate, or chondroitin sulfate. In a preferred embodiment, the sugar compound is a pharmaceutically acceptable salt or ester of N-acetylglucosamine. However, N-acetylglucosamine is derived from shellfish and some consumers can have allergic or hypersensitivity reactions to this compound which include skin rashes, itching, nasal congestion, wheezing and a tingling sensation in the mouth. During severe cases, hypersensitivity reactions also called anaphylaxis can become fatal and life threatening. Moreover, there is no mention of the use of either vitamin D3 or silica.

U.S. Pat. No. 6,149,933 teaches that a dietary supplement can be used to retard, prevent, suppress, and/or even reverse the graying of hair. For some people whose hair has already turned gray, their natural hair color can be restored, and maintained, naturally without the use of dyes, colorants, or the like. The dietary supplement contains, in effective amounts, a copper salt; para-aminobenzoic acid (PABA) or salts thereof; pantothenic acid or salts thereof; and vitamin B6 in an appropriate carrier.

U.S. Pat. App. No. 20110287061 teaches that a dietary supplement product can be used to reverse the graying of hair. A dietary composition for promoting melanogenesis in hair of a user comprising approximately 5,000 IU per day of catalase, a synergistic core component, 5-alpha-reductase inhibitors, an antioxidant component, and approximately 200 mg per day of L-tyrosine. Preferably, the synergistic core component comprises copper, vitamin B6, biotin, para-aminobenzoic acid, folic acid, zinc, and pantothenic acid. Furthermore, 5-alpha-reductase inhibitors preferably comprise saw palmetto, phytosterols, and nettle root extract. The antioxidant component preferably comprises horsetail extract (Equisetum arvense), Fo-Ti root (Polygonum multiflorum), chlorophyll, and barley grass.

U.S. Pat. App. No. 20110236493 teaches the use of porous materials for a variety of applications. The composition consists of a mesoporous microencapsulation material selected from one or more of silicon, silicate, silica, or from fumed silicon dioxide as well as a capping layer selected from one or more of titanium dioxide, carageenan, xanthum gum, cellulose gum, tocopherol, cocoa butter, vegetable fat, milk fat, lecithin, glyceryl oleate, seed oil, cyclopentasiloxane, or paraffin. This patent application describes the use of a porous material in the preparation of various food products (i.e., beverage or non-beverage), consumer care compositions selected from an oral hygiene composition, a hair care composition, a skin care composition or a cosmetic composition. The present invention extends to said hair care compositions for use in the prevention and/or treatment of dandruff and/or split ends and/or head-lice and/or hair-loss. However, this patent application deals with the topical application of hair care products or the ingestion of beverages or the use of oral hygiene solutions.

While this patent application discusses the fact that vitamins, including vitamin D3 can be encapsulated into silica, it does not discuss the use of higher doses of vitamin D3 specifically for the purpose of treating hair loss and hair thinning. Furthermore, a capping layer is not needed in this current patent application because it is preferable that the vitamins are released quickly inside the body. The capping layer applied over the silica microencapsulates finds more utility in a topical application on the scalp or skin. Additionally, this present invention focuses on the use of silica in several key areas different from the 20110236493 application. In this case, the silica functions as a microencapsulating agent, but also as an oil slurry viscosifier, and as a nutrient to support healthy hair growth and retention. The final application is also not a food product nor an oral hygiene solution nor a topical application for the skin and/or scalp. It is an oil-filled hard or soft gelatin capsule suitable for oral administration in humans.

U.S. Pat. App. No. 20100172993 teaches compositions having particles comprising inorganic element(s); one or more active ingredient and optionally a release rate modulating agent, suitable for the delivery of active ingredients to human and animal tissues. The particles are nanoparticles or microparticles or mixtures thereof, made preferably by a sol-gel method. The compositions are useful for application to the topical or mucosal surfaces preferably in the form of creams, gels, lotions, dry powders, spray, foam and other suitable forms. The inorganic element is selected from group comprising of silica, alkaline metals, alkaline earth metals, transition metals, especially zinc, calcium, magnesium, titanium, silver, aluminium, and lanthanides, their salts, hydrates, as well as combinations thereof. This patent application mentions the use of a number of active ingredients for topical delivery to the skin and other mucosal surfaces. It does not specifically mention the use of the fat-soluble vitamins (i.e., A, D, and E) in the oral treatment of hair loss and hair thinning. Moreover, this invention relates to microparticles or nanoparticles prepared by a sol-gel synthesis method or any of its modifications known in the art.

The nanostructures of the present invention produced by sol-gel processes is generally comprised of the following steps: preparation of a solution or suspension, of a precursor formed by a compound of the element (M) forming the oxide or alkoxide; hydrolysis (acid or base catalyzed), of the precursor, to form M-OH groups. The so obtained mixture, i.e. a solution or a colloidal suspension, is named sol; polycondensation of the M-OH or M-OR groups according to the reactions M-OH+M-OH→M-O-M+H₂O and M-OR+M-OH→M-O-M+ROH characterized by an increase of the liquid viscosity (gelation) and by the contemporaneous formation of a matrix called gel.

U.S. Pat. App. No. 20070231377 teaches compositions and methods for treating hair loss, nail brittleness and skin conditions, and for promoting or enhancing hair growth. The composition generally includes at least one methionine analog or derivative. The hydroxyl analog of methionine is a metal chelate. The metal ion is selected from the group consisting of zinc ions, copper ions, manganese ions, iron ions, chromium ions, nickel ions, cobalt ions, silver ions, selenium ions, magnesium ions and calcium ions. The route of patient administration is via oral, injection, or topically.

The present invention addresses the need in the art for a composition of active molecules, i.e., fat-soluble vitamins, which are better protected from oxidation. Fumed silicon dioxide is a highly porous material and can incorporate and protect vitamins A, D, and E during long-term storage. Sonication can be used to remove dissolved air as well as to drive the fat-soluble nutrients inside of the porous silica nanoparticles. The incorporation of these vitamins into the porous silica also gives this preparation certain controlled-release properties. This is beneficial for prolonging the effectiveness of the vitamin E mixed tocotrienols since they are known to have a much shorter biological half-life as compared with alpha-tocopherol. For example, it has been reported by Yap et al., 2001; J. Pharm. Pharmacol. Jan.; 53(1):67-71 and Schwedhelm et al., 2003; Clin. Pharmacokinet. 42(5):437-459, that while the half-life for alpha-tocopherol is between 73- 81 hours, the half-lives for the mixed vitamin E tocotrienols range from only 2.3-4.4 hours.

It has been previously shown that vitamin C (ascorbic acid) itself an antioxidant, can help protect other nutrients such as vitamin E from oxidative damage. There is increasing evidence that the combination of vitamins C and E act synergistically with each other (Steenvorden and van Henegouven 1997; J. Photochem. Photobiol. B Biol. 41:1-10).

There remains a yet unmet need for an all-natural product containing several nutritional ingredients in the form of fat-soluble vitamins and the inorganic trace mineral, silicon, which are both useful for preventing hair loss and also stimulate the growth of new hair follicles in humans. This dosage form should also be well-tolerated and have minimal side-effects. This dosage form is also a reasonable size to improve patient swallowability and compliance. Currently, there is no vitamin D-based dosage form which is marketed for these purposes.

Health Benefits of Vitamin D3

Very few foods in nature contain vitamin D. The flesh of fatty fish (i.e., salmon, tuna, and mackerel) and fish liver oils are among the best sources while small amounts of vitamin D are found in beef liver, cheese, and egg yolks. Vitamin D3 is required throughout the lifespan. Not only is it needed for the proper formation of bones but it has also been found to play a role in a number of other important physiologic systems. Vitamin D3 facilitates calcium and phosphate absorption and retention and enables normal mineralization of bone. By modulating blood levels of calcium, vitamin D3 influences the development of vascular and tissue calcification, which can lead to damage to the heart, blood vessels, and kidneys. Laboratory and animal evidence as well as epidemiologic data suggest that vitamin D3 status could also affect cancer risk. Emerging epidemiologic data suggest that vitamin D3 may have a protective effect against colon cancer, but the data are not as strong for a protective effect against prostate and breast cancer, and are variable for cancer at other body locations.

Vitamin D3 deficiency can result from an inadequate dietary intake coupled with inadequate sunlight exposure, and other disorders that can limit its absorption. A deficiency results in impaired bone mineralization, and leads to bone softening diseases (i.e., rickets in children and osteomalacia in adults). Together with calcium, vitamin D3 also helps protect older adults from osteoporosis. However, sunlight exposure, to avoid deficiency, carries other inherent risks such as the development of skin cancer. The risk of melanoma is reduced by the adequate ingestion of vitamin D3 containing foods in combination with dietary supplements. Furthermore, in certain locations of the world, i.e., particularly the northern and southern regions of the planet, the prolonged winter season does not allow people to obtain adequate sunlight needed throughout the year.

Hair Loss

Hair follicle cells have a high turnover. Their active metabolism requires a good supply of nutrients and energy. Hair loss in men and women has many genetic and physiological factors. The most common form of balding is due to an auto-immune disorder known as androgenetic alopecia, which is also known as “male pattern baldness,” and which affects over 50% of Caucasian men. Other non-genetic events which can also lead to hair loss include pregnancy and childbirth; menopause; severe emotional stress; rapid or profound weight loss; malnutrition; prolonged illness; chemotherapy-induced alopecia; and surgery. While several dietary supplements and topical formulations are commercially available for the treatment of hair loss and the promotion of hair growth, none of the current so-called “hair growth stimulants” have proven to be very efficacious. Furthermore, some of the prescribed drug therapies also carry their own inherent risks and side effects.

It has been known for some time that the complete loss of hair is associated with the finding that the vitamin D receptors (VDR) are normally found in hair follicles (Stumpf et al., Science 1979, 206:1188). Hair follows a specific growth cycle with three distinct and concurrent phases: anagen, catagen, and telogen. Each phase has specific characteristics that determine the length of the hair. All three phases occur simultaneously; one strand of hair may be in the anagen phase, while another is in the telogen phase. The anagen phase is known as the growth phase and can last between two and six years. About 85% of the hairs on one's head are in the anagen phase at any given time. Signals sent out by the body determine when the anagen phase ends and the catagen phase begins. The catagen phase, also known as the transitional phase, allows the hair follicles to renew themselves. During the telogen, or resting, phase the hair and follicle remain dormant anywhere from 1-4 months. Ten to fifteen percent of the hairs on one's head are in this phase of growth in any given time. The anagen phase begins again once the telogen phase is complete. The preceding hair strand is pushed up and out by the new, growing strand in a process known as shedding. Telogen effluvium is a scalp disorder characterized by the thinning or diffuse shedding of hair resulting from the early entry of hair into the telogen or resting phase. A physically or emotionally stressful triggering event (i.e., childbirth, major surgery, chronic illness, hormone changes, emotional disorders, crash diets) can precipitate telogen effluvium two to three months after the metabolic or hormonal insult. Hair loss often resolves spontaneously within about six months, but can also become chronic telogen effluvium. Hair disorders can cause serious psychological distress in both men and women, adversely affecting their self--esteem and quality of life.

Vitamin D3 exerts its biological actions by binding to the VDR. Alopecia is a feature of VDR mutations in humans and in VDR-null mice. The alopecia results from an inability to initiate the anagen phase of the hair cycle after follicle morphogenesis is complete. Thus, once the initial hair is shed it does not regrow. VDR expression in the epidermal component of the hair follicle, the keratinocyte, is critical for maintenance of the hair cycle (Skorija et al., Mol. Endocrinol. 2004; 19(4):855-862). Moreover, Kong et al., (J. Investig. Dermatol. 2002; 118(4): 631-638) targeted human VDR expression (hVDR) in the skin of transgenic mice and showed that alopecia in knockout (VDR^{-/-}) mice was reduced, and the rate of anagen follicle formation was accelerated.

### SUMMARY OF THE INVENTION

Evidence exists for the regulation of the hair cycle via biologically active vitamin D3 binding to VDR. Furthermore, hair growth provides a shield against ultraviolet-induced skin damage and cancer in mammals (a function of VDR that facilitates healthy aging).

Vitamin D3 is involved in the regulation of many biologic processes inside the body. It is also strongly believed that the majority of people are vitamin D3 deficient. The ingestion of therapeutic levels of vitamin D3 (i.e., 3,000-5,000 I.U. per day) can improve the quality of life and also delay the progression of other age-related illnesses. The dosage form is also convenient to administer, thus improving patient compliance. In addition, the other ingredients present in the formulation can also act synergistically to maintain the integrity of the existing hair follicles as well as to prevent further hair loss.

The other supporting nutrients consist of two other fat-soluble vitamins, vitamin A and vitamin E. Both are microencapsulated in the pores of the silica. The silica has several roles, it acts as a microencapsulator for the fat-soluble vitamins, it acts as a viscosifier to form an oil slurry, and it also serves as a supplemental nutrient to nourish the hair follicles.

### DETAILED DESCRIPTION OF THE INVENTION

Vitamin D is a group of fat-soluble pro-hormones, the two major forms of which are vitamin D2 (or ergocalciferol) which is produced by some kinds of phytoplankton, yeasts, and higher fungi such as mushrooms, and vitamin D3 (or cholecalciferol or calcitriol). The term vitamin D as used herein also refers to metabolites and other analogues of these substances. Vitamin D obtained from sun exposure, food and supplements is biologically inert and must undergo two hydroxylations in the body to get converted into the active form. The starting material, 7-dehydrocholesterol is converted in the skin (i.e., epidermal keratinocytes) following exposure to sunlight, specifically ultraviolet B radiation (290-315 nm), to 25-hydroxyvitamin D. This first hydroxylation reaction occurs in the liver. The second hydroxylation reaction occurs primarily in the kidney and results in the formation of the physiologically active 1,25-dihydroxyvitamin D, also known as calcitriol.

The vitamin D council, a 501(c)(3) non-profit organization founded in 2003 and dedicated to informing the general public on the health benefits of vitamin D, has stated that the U.S. Government's recommended Adequate Intake for vitamin D3 is too low to receive many of its health benefits. The government's tolerable upper intake level was set at 2,000 I.U./day but has recently been changed to 4,000 I.U./day. According to Heaney et al., Am. J. Clin Nutr. (2003) 77:204-210, healthy men seem to use between 3,000-5,000 I.U. of vitamin D3 per day and this apparently meets more than 80% of their winter vitamin D3 need with cutaneously synthesized accumulations from solar sources during the preceding summer months. The amount of vitamin D3 that a person naturally produces in their skin from sun exposure is approximately 10,000 I.U. (Vieth, J. Bone Miner. Res. 2007 December; 22 Suppl. 2, V:64-68), therefore, a daily intake of 5,000 I.U. derived from diet and/or supplements is well within the safe range for a majority of the population. Very high levels of vitamin D3 (above 10,000 I.U. per day) are known to cause hypercalcemia as well as kidney and tissue damage.

This nutritional supplement is to be administered in a single unit dosage form. As used herein, single unit dosage form shall mean a dosage form wherein all of the micronutrients of the composition are in a single pill, tablet, caplet, capsule, quick dissolving tablet, orally disintegrating tablet (ODT), chewable tablet, soft gelatin capsule, standard two-piece hard gelatin capsule, effervescent tablet, powder, liquid suspension, and food product. It however is recognized that the single unit dosage form may be administered as a single dose (i.e., taking 1-2 capsule(s) per day); or in multiple doses. Preferably the dosage form is administered as 1 dose per day.

These compositions can be manufactured using conventional equipment and techniques known in the art. When preparing dosage forms incorporating the compositions of the invention, the nutritional components are normally blended with different diluents or excipients such as oils (i.e., soybean and cottonseed oils) and the like; and emulsifiers such as lecithin, polysorbate 80, and the like; preservatives, such as propylparaben, methylparaben, and sodium benzoate; and colorants such as FD&C dyes and the like. The oral dosage forms may further contain sweetening and/or flavoring and/or coloring agents.

Diluent oils would include soybean oil, corn oil, sunflower oil, canola oil (rapeseed), safflower oil, cottonseed oil, olive oil, sesame oil, peanut oil, medium chain triglycerides (MCT) oil such as capric and caprylic triglycerides and other oils in the Miglyol™ series, grape seed oil, calendula oil, avocado oil, coconut oil, wheat germ oil, sweet almond oil, and mixtures thereof can be included in the present invention.

The vitamins A, D, and E are fat-soluble and are microencapsulated in the pores of the silica and dispersed in the oil phase homogeneously. Emulsifiers such as lecithin, glycerol monostearate, fatty acid mono- and diglycerides, glycerol triacetate, propylene glycol monostearate, polysorbates (i.e., polyoxyethylene sorbitan monooleate) also known as “Tweens”, or the sorbitan esters, also known as “Spans” and mixtures thereof can also be included in the composition of the present invention as necessary at levels ranging from 1.0% to about 50.0% (w/w).

Vitamin E is a generic name for a family of four isomers of tocopherols and four isomers of tocotrienols. All eight isomers have a 6-chromanol ring structure and a side chain. There are 4 tocopherols (α, γ, β, and δ) with a fully saturated side chain. The four tocotrienols (α, γ, β, and δ), although structurally similar to tocopherols, have unsaturated side chains with double bonds at the 3′, 7′, and 11′ positions in the side chain, individual tocopherols and tocotrienols differ from each other in the number and position of methyl groups in the aromatic chromanol ring. All isomers of Vitamin E exert a wide spectrum physiological effect. For example in addition to being antioxidants, tocotrienols have been shown to he potent suppressers of B16 melanoma cell proliferation in vitro, and γ-tocotrienol has been shown to inhibit the growth of human breast cancer cell growth in vitro more effectively than the popular breast cancer drug, Tamoxifen.

A tocotrienol is a more effective antioxidant than a tocopherol because its unsaturated side chain facilitates better penetration into saturated fatty layers of the brain and liver. Tocotrienols can lower the incidence of tumor formation, DNA damage, and cellular damage. No individual tocotrienol out of the four is most potent. Each isomer of tocotrienol works best in different parts of the body and furthermore, all of the isomers work synergistically as a team to confer the maximum health benefits. Only palm tocotrienol complex provides all the four forms of tocotrienols (alpha, beta, gamma and delta-tocotrienol).

Vitamin E is involved in the metabolism of all cells. It acts as an effective antioxidant by scavenging and neutralizing free radicals before they have a chance to cause cellular damage. This vitamin contributes to hair health via its blood flow enhancing effects. Vitamin E nourishes the hair follicles as well as stimulating the scalp with oxygen and increased blood flow. Vitamin E is the generic term for a group of related substances that include the four tocotrienol isomers, alpha-tocotrienol, beta-tocotrienol, delta-tocotrienol, and gamma-tocotrienol and/or the four tocopherol isomers, alpha-tocopherol, beta-tocopherol, delta-tocopherol, and gamma-tocopherol. Furthermore, each of these four compounds has a “d” form, which is the natural form, and a “dl” form, which is synthetic. In the present invention, vitamin E would be in the “d” or natural form. In addition, the form of vitamin E can be as tocotrienol succinate, tocotrienol acetate, tocopherol succinate, tocopherol acetate, or mixtures thereof. In a preferred embodiment the effective amount of vitamin E tocotrienols is about 30 mg (44 I.U.) per day.

Vitamin A is a fat-soluble vitamin containing retinols and beta carotene and also functions as an antioxidant. Vitamin A lubricates the hair roots, promoting natural shine and healthy hair follicles. In a preferred embodiment of the invention, the effective amount of vitamin A is between 3,000-5,000 I.U. per day.

Silicon is a trace mineral naturally present in the body in various tissues, body fluids, bones, and hair. In bones, it helps to maintain collagen and it is one of the building blocks of hair. Human hair contains approximately 100 μg/g of silicon. Silicon has been shown to reduce hair loss, increase hair elasticity, and to decrease the problem of spilt-ends. The outer shaft of hair, which provides elasticity and strength, is rich in silicon. Hair that contains a larger amount of silicon tends to fall out less and have more shine and luster.

The silica derived from fumed silicon dioxide has several important physical properties which are important to this nanoparticulate formulation. This grade of fumed silicon dioxide has a very high BET surface area between 175-225 m²/g. This enables lower quantities to be used in the formulation to achieve the same results. The average particle size is 12 nanometers and this grade is very porous, which enables it to absorb and act as a carrier for the fat-soluble vitamins. Moreover, the moisture content is very low (loss on drying less than 1.5%). Utilizing low moisture excipients is vital when formulating gelatin-based capsule formulations. Moisture acts to cause leaking and rupture of the gelatin shells during storage.

Colloidal silicon dioxide is typically used as a flowability aid for tablets and hard gelatin capsules. Part of the novelty of this invention is the fact that it is typically not used in oil-based formulations for gelatin capsules as a viscosifier or microencapsulating agent.

The delivery system for this product will consist of some type of capsule. The capsules can be enteric-coated (release >pH 5.5), in order to bypass the stomach and release the fat-soluble vitamins in the duodenum. Furthermore, the capsules can be either in the form of soft gelatin capsules or specially designed standard two-piece capsules (i.e., Licaps®), which can be composed of either gelatin or hydroxypropylmethylcellulose (HPMC), and which are designed to contain a liquid fill. An advantage of using Licaps® over soft gelatin capsules is that they do not contain any plasticizers and therefore, no “plasticizer channels” are present. Thus, Licaps® have a lower oxygen permeability rate which protects the capsule contents against oxidation and prolongs stability.

The compositions are useful for the prophylaxis and treatment of hair loss in humans. The compositions are useful in treating children or adults experiencing hair loss, including those patients receiving various oncology-related therapies. All of the ingredients used herein are approved by the FDA and already have wide consumer acceptance.

The compositions provided by the present invention are capable of enhancing the actual density of human hair, as can be determined by the actual number of hairs per area on the scalp; and may also increase the keratinization of the hair.

Moreover, in addition to the inactive ingredients described herein, the compositions preferably comprise additional micronutrients to supplement the daily diet.

The following examples are intended to illustrate, but not to limit, the invention.

### EXAMPLE 1

A dietary supplement containing the following ingredients was prepared in a capsule form. Soybean and corn oils were used as diluents for the fat-soluble vitamins, A, D, and E. The standard dosage is two capsules taken orally once per day.

| Ingredient | Amount in Capsule (mg) | Daily Dosage (mg) |
|---|---|---|
| Soybean oil | 149.0 | 298.0 |
| Corn oil | 124.0 | 248.0 |
| Vitamin C (ascorbic acid) | 40.0 | 80.0 |
| Vitamin E tocotrienols | 15.0 | 30.0 |
| (mixed isomers) | | (44 I.U.) |
| Colloidal Silicon dioxide | 7.0 | 30.0 |
| | | (14 mg as Silicon) |
| Vitamin A | 0.45 | 0.9 |
| | | (3000 I.U.) |
| Vitamin D3 | 0.063 | 0.125 |
| | | (5000 I.U.) |

### EXAMPLE 2

A dietary supplement containing the following ingredients was prepared in a capsule form. Only soybean oil was used as a diluent for the fat-soluble vitamins, A, D, and E. The standard dosage is two capsules taken orally once per day.

| Ingredient | Amount in Capsule (mg) | Daily Dosage (mg) |
|---|---|---|
| Soybean oil | 273.0 | 546.0 |
| Vitamin C (ascorbic acid) | 40.0 | 80.0 |
| Vitamin E tocotrienols | 15.0 | 30.0 |
| (mixed isomers) | | (44 I.U.) |
| Colloidal Silicon dioxide | 7.0 | 30.0 |
| | | (14 mg as Silicon) |
| Vitamin A | 0.45 | 0.9 |
| | | (3000 I.U.) |
| Vitamin D3 | 0.063 | 0.125 |
| | | (5000 I.U.) |

While the invention has been described with respect to certain specific embodiments, it will be appreciated that many modifications and changes may be made by those skilled in the art without departing from the invention. It is intended, therefore, by the claims to cover all such modifications and changes as may fall within the true spirit and scope of the invention.

### Definitions

The term “mixed isomers” of vitamin E tocopherols and/or tocotrienols refers to a mixture of the four isomers, namely, d-alpha, d-beta, d-gamma, and d-delta.

The term “biological half-life” or elimination half-life of a substance is the time it takes for a substance (e.g., a metabolite, drug, radioactive nuclide, or other substance) to lose half of its pharmacologic, physiologic, or radiologic activity. Biological half-life is an important pharmacokinetic parameter and is usually denoted by the abbreviation t_{1/2}.

An emulsifier or surfactant lowers the interfacial tension between the water-phase and oil phase to allow the two phases to be miscible and stable on storage. Examples include soy lecithin, mono- and diglycerides of fatty acids, glycerol triacetate, propylene glycol monostearate, the polysorbates (i.e., polyoxyethylene sorbitan monooleate) also known as “Tweens,” or the sorbitan esters, also known as “Spans,” and mixtures thereof.

1. A nutritional supplement for providing nutritional support for the mitigation of hair loss and hair thinning in men and women and comprising: an effective amount of vitamin D3; an effective amount of vitamin A; an effective amount of vitamin E as tocopherols and/or tocotrienols (mixed isomers); and an effective amount of silica (colloidal silicon dioxide); wherein the integrity of the hair follicles and associated glands are supported. Other supportive antioxidant nutrients such as vitamin C (ascorbic acid) may also be included.

2. A composition according to claim 1, further comprising at least one fat-soluble nutrient being a member selected from the group consisting of vitamin A, and vitamin E (as mixed isomers of tocopherols and/or tocotrienols).

3. A composition according to claim 1, further comprising several water soluble nutrients selected from the group consisting of biotin, zinc citrate, vitamin C (ascorbic acid), L-methionine, L-cysteine, niacin, and D-calcium pantothenate (pantothenic acid).

4. The nutritional supplement of claim 1, wherein the effective amount of one of the active agents, vitamin D3, is between 3,000 to 5,000 I.U. per day.

5. A composition according to claim 1, wherein the effective amount of one of the active agents, vitamin A, in the form of retinyl acetate or retinyl palmitate, is between 1,000 I.U. to about 5,000 I.U. per day.

6. A composition according to claim 1, wherein the effective amount of one of the active agents, vitamin E (as mixed isomers of tocopherols and/or tocotrienols), is between about 15 mg (22 I.U.) to about 30 mg (44 I.U.) per day.

7. A composition according to claim 1, wherein the effective amount of one of the active agents, silica (colloidal silicon dioxide), is between 5 to 15 mg per day.

8. A composition according to claim 1, wherein the microencapsulation material is selected from one or more of silicon, silicate, or silica.

9. A composition according to claim 1, wherein the microencapsulation material is colloidal silica derived from fumed silicon dioxide. The amount of silica used as an oil viscosifier is between 0.5 to 6.0%.

10. A composition according to claim 1, wherein the microencapsulation material is silica derived from fumed silicon dioxide and has an average particle size between 10 nanometers and 40 nanometers.

11. A method of making composition according to claim 1 comprising: (a) heating the oil(s) to between 40-50° C. and subsequently dissolving the microencapsulating agent into the oil(s) to form a solution; (b) dissolving the active ingredient/s (i.e., fat-soluble vitamins) and other supporting nutrients (i.e., ascorbic acid) into the oil(s) to form a slurry; (c) sonicating the oil slurry to remove dissolved air and to incorporate the fat-soluble vitamins into the porous structure of the colloidal silica; (d) filling the oil slurry into the capsules to produce the dosage form.

12. A method for reducing or preventing hair loss or hair thinning in human subjects comprising the step of administering to said human a formulation comprising vitamin D3 as one of the active agents, wherein the administering of said nutritional formulation is carried out so that the overall dose of said active agent is in the range of from about 3,000 I.U. to about 5,000 I.U. of total vitamin D3 per day.

13. A method according to claim 11, wherein the step of administering is carried out by consuming one dose (e.g., two capsules) during the day.

14. A method according to claim 11, wherein the step of administering is carried out for a period of from about 4 to about 26 weeks.
